(19) United States
(12) Patent Application Publication
(10) Pub. No.: US 2011/0035023 A1
(43) Pub. Date: Feb. 10, 2011

(54) PROSTHESIS FOR PROMOTING THE IN VIVO RECONSTRUCTION OF A HOLLOW ORGAN OR A PORTION OF A HOLLOW ORGAN

(76) Inventors: Veronique Maquet, Berloz (BE); Sandrine Gautier, Liege (BE); Very Coulic, Marcinelle (BE)

Correspondence Address: CLARK & BRODY, 1700 Diagonal Road, Suite 510, Alexandria, VA, 22314, US

(21) Appl. No.: 12/675,510

(22) PCT Filed: Sep. 1, 2008
(86) PCT No.: PCT/EP08/61473

§ 371 Date: Oct. 28, 2010

(30) Foreign Application Priority Data
Aug. 31, 2007 (FR) 0757308

(51) Int. Cl. A61F 2/04; A61B 17/00; A61K 9/00; A61P 43/00

(52) U.S. Cl. 623/23.65; 623/23.71; 623/23.66; 128/898; 424/426

(57) ABSTRACT

The invention relates to a prosthesis for promoting the in vivo reconstruction of a hollow organ or of a portion of a hollow organ, characterized in that it comprises:
a biodegradable hollow tubular support membrane comprising at least one biocompatible and biodegradable polymer material, said support membrane being constituted of a porous outer layer and an essentially non-porous inner layer; and
a material of living biological origin at the outer surface, and/or within at least one portion of the porous layer of said support member, and/or over the surface of the essentially non-porous layer facing the porous layer, said material of biological origin being chosen in order to allow the in vivo reconstruction of said organ or of said organ portion.

The invention relates to a method for producing such a prosthesis and the medical applications thereof, especially for reconstructing at least one portion of a hollow tubular organ, in particular an esophagus.

The present invention relates to a novel prosthesis for promoting the in vivo reconstruction of a hollow organ or a portion of such an organ.

It relates more specifically to a bioprosthesis for the in vivo reconstruction of a human or animal hollow organ, or a portion of such an organ.

### PRIOR ART

The replacement of hollow tissues and in particular that of circular defects of such organs and more specifically of the esophagus remains one of the most difficult problems in surgery, especially in digestive surgery.

Up until the 1950s, autologous segments (taken from the patient him/herself) of intestines and stomachs were widely used for replacing hollow organs such as segments of esophagus, of common bile duct, the bladder, the urethra or for the rechanneling of the Fallopian tubes. These autografts are however associated with a high percentage of postoperative complications.

In the 1960s, owing to the development of polymers, polymer prostheses were widely used for various applications (esophagus, stomach, biliary duct, blood vessels). These prostheses are made from various materials (polyethylene, silicone, polyurethane, acrylate-amide terpolymer, polytetrafluoroethylene). These polymer prostheses are usually well tolerated but their integration is not optimal. Since the prosthesis is only in contact with the living tissues over a single face, the colonization thereof is not achieved, which results in the formation of eschar, in its detachment and its elimination. Whatever their composition, these prostheses are therefore only temporary and must be replaced regularly. Their clinical use is therefore limited to certain applications such as the drainage of the common bile duct, pancreatic ducts, the tracheal tubes and esophageal tubes.

The life expectancy of patients suffering from advanced esophageal cancer is very short and the care of most of these patients is limited to palliative treatments: surgical resection, radiotherapy/chemotherapy, with very mediocre results. Although the use of prostheses has been proved to be effective for resolving dysphasia and improving the quality of life of patients suffering from esophageal cancer, complications such as migration, perforation and obstruction by food leads to mortalities that are too high.

Bioprostheses have been designed to cover the inner surface of artificial tubes with tissue cultures. This option has proved effective for the replacement of partial lesions, in the form of types of dressings, often known as “patches”, but have not been used for resolving circular lesions of the esophagus.

The use of expandable metallic stents is considered to be an affective alternative to non-expandable plastic tubes, but remains burdened by the same complications (eschars, elimination).

Patches represent an effective therapeutic solution for the treatment of partial lesions that do not cut into the entire circumference of the organ (for example, after ablation of diverticula). However, currently the therapeutic options for curing the cellular lesions such as those that appear on the esophagus following cancer or a burn with severe stenosis are very limited and should be based on a better design of the esophageal prosthesis.

Strategies based on biodegradable polymers have appeared as an alternative for the purpose of developing or regenerating new tissues. Many biodegradable materials in the form of sponges, mesh fabrics (known as “meshes”, or matrices in certain cases), tubes and nanofibers have been used, in rats or mice, as a support member for regeneration of the esophagus, in experimental models. In order to do this, use has been made of synthetic polymers such as biodegradable polyesters from the polylactic acid (PLA), polyglycolic acid (PGA) and polycaprolactone (PCL) family. Some of these products are commercially available (e.g. Vicryl surgical mesh).

However, these synthetic polymers alone are not capable of inducing the biological response that leads to the regeneration of tissues, due to a lack of biomimetism, which often necessitates recourse to surface modifications (grafting of collagen, or fibronectin).

The transplantation of adult tissues (duodenal mucosa and submucosa revascularized ileal grafts, lyophilized dura mater) and autologous materials (cells, mucosa) has been described for treating esophageal lesions.

However, adult tissues do not withstand ischemia, which limits their chance of survival after transplantation. Grafts of adult tissues are also difficult, incapacitating and require repeated interventions. They are one option for closing up non-circular defects but not for circular lesions.

The total transposition of the stomach has also been described, but it generates problems such as reflux, and too rapid evacuation to the intestine.

Combined strategies based on the use of synthetic prostheses combined with autologous cells rather than adult tissues have been proposed.

This concept, known as tissue engineering, has aroused great interest in the last 10 years.

It is based on the use of a natural or synthetic biodegradable polymer support member combined with human, preferably autologous, cells, precultured in vitro. The cells/matrices assembly is then implanted in vivo for the purpose of reconstructuring, regenerating or repairing a damaged organ or tissue. This strategy was developed by Marzaro et al. (Journal of Biomedical Materials Research, 2006, 77a(4), 795-801), who proposed the use of a homologous esophageal acellular matrix and autologous smooth muscle cells in vitro for the development of an implantable esophagus.

Two-layer tubes composed of seeded collagen networks and of a muscle layer have also been manufactured for esophageal engineering. They allowed cell infiltration and neovascularization.

Use has also been made of a decellularized esophagus as a biocompatible support member for tissue engineering. However, this option raises the problem of the availability of the tissues, and also the use of the prosthesis in order to correspond to the size and dimensions of the organ to be repaired. Furthermore, the artificial support members and the autologous tissues used for the reconstruction of the esophagus may induce complications like stenosis and leakage in the long term since their inner surface cannot be entirely covered with epithelium.

Chinese patent application CN 1410034 A describes a support member having a two-layer structure composed of a skin and a subdermal layer containing vascular cells for the tissue engineering of the esophagus. The support member may be a biomaterial or a synthetic material, but is preferably combined with an acellular matrix. The seeded cells may be fibrocytes, endothelial cells or keratinocytes. The device could be used for tissue regeneration, but not for the replacement of a complete organ such as the esophagus.

All the other cases of therapy that currently exist have problems of cell collection, design time and high cost.

Patent application WO 2006/047758 describes a way of preparing chitosan tubes comprising a porous layer and a non-porous layer. This patent application describes the use of centrifugal force as a means for producing a tubular structure, and inevitably positions the non-porous layer on the outer surface of the tubular structure. This application does not therefore describe a tubular structure that makes it possible to solve the technical problem of reconstructuring a tubular organ. Moreover, an example of tissue reconstruction is not given.

### OBJECTIVE OF THE INVENTION

The objective of the invention is to solve all of the problems described above, especially by providing a novel prosthesis for promoting the reconstruction of a hollow organ or a portion of a hollow organ.

Within this context, the invention proposes to solve the technical problem of withstanding the movements imposed on the organ to be reconstructed, and especially on the esophagus which is partly located in an area of the body that is regularly in motion (twisting, swallowing, etc.): the neck.

Moreover, the objective of the invention is to solve the problem that consists in providing a prosthesis that has suitable properties for reconstructuring the organ, such as the mechanical strength and/or the leaktightness of the prosthesis to a bodily fluid that may or may not be in permanent contact with the inner surface of the organ to be reconstructed.

### DESCRIPTION OF THE INVENTION

Chitosan is a biopolymer obtained by deacetylation of chitin, which is present in the wall of crustations, the cuticle of arthropods, the endoskeletons of cephalopods, diatomaceous earths, or else of fungal origin such as in the walls of fungi. It possesses advantageous properties including biocompatibility, biodegradability and a structure similar to the glycosaminoglycans of the extracellular matrix. Chitosan is of great interest for biomedical applications including wound healing, systems for the controlled release of medicaments, hemostatic devices, surgical applications (resorbable suture threads, anti-adhesion barriers), ophthalmology and applications in tissue engineering, cell encapsulation, gene therapy and vaccination.

A review of the potential applications of chitosan was published by Khor and his collaborators [Khor and Lim, Biomater 2003; 24: 2339-2349].

Chitosan is suitable for tissue engineering of the conceived hollow organs when it is in the form of a porous cellular structure. In “Chitin-based tubes for tissue engineering in the nervous system”, Biomater 2005; 26-4624-4632, Freier and his collaborators reported a method for preparing chitosan tubes obtained from an alkaline hydrolysis of chitin. The authors demonstrated a cytocompatibility of the chitosan films with dorsal root ganglion neurons and neural growth in vitro.

WO 2007/042281 A2 describes a method based on a process for the extruding of an N-acylchitosan gel for the construction of chitosan tubes and fibers and derivatives of chitosan that have sufficient mechanical strength, without the use of toxic solvents or of crosslinking agents and other toxic compounds.

Methods for preparing specific structures containing chitosan, such as hollow tubes having a porous structure, have been described by Madihally and his collaborators [Madihally and Matthew, Biomater 1999; 20:1133-1142]. The porous tube support members are prepared by freezing a chitosan solution contained in cylindrical plastic tubes. Tubes with a non-porous luminal membrane may be obtained by a first coating of the inert tube with a chitosan film, said film being obtained by gelation of chitosan in a basic medium followed by its dehydration in air. After drying and rehydrating the support member using a sodium hydroxide or ethanol treatment, and also a neutralization with a saline-phosphate buffer, the support member is characterized by electron microscopy and by mechanical tests. Several biological evaluations of the support member were carried out, but this document does not present any technical result in a tissue engineering application.

The feasibility of use of chitosan-based materials for developing a tissue-engineered esophagus has been studied by Qin and his collaborators (Qin, Xiong, Duier Junyi Daxue Xuebao 2002, 23, 1134-1137), who implanted collagen-chitosan membranes with rat esophageal epithelial cells in a submuscular manner. They showed that the grafted assembly remains healthy after 2 weeks and is completely degraded after 4 weeks following implantation. The authors showed a cellular compatibility of the polymeric support member but do not give other descriptions of the support member, and in particular do not describe the use of this support member as a biodegradable artificial esophagus.

In view of the problems posed by the reconstruction of hollow organs and, very particularly of the esophagus, especially when they are affected by circulation defects, it appears that there is a real need to develop novel solutions capable of allowing the regeneration of these organs, especially when they are affected by circular defects.

The present invention proposes a novel type of prosthesis or bioprosthesis which can be implanted in or on, or in relation to, a hollow organ and, very particularly, the esophagus, with a view to ensuring its regeneration.

This bioprosthesis comprises a biodegradable porous support member combined with a living material, preferably that is not very differentiated or is undifferentiated and, preferably a fetal material, or combined with tissue cells of the organ to be reconstructed.

The biodegradable porous support member is advantageously constituted of chitosan, but may also be constituted of any biodegradable and biocompatible polymer material capable of being used in order to result in the desired porosity. A combination of various porous support members is also covered by the present invention (such as, for example, chitosan/collagen, chitosan/glycosaminoglycans such as chitosan/hyaluronic acid combinations, or any other combination well known to a person skilled in the art).

The biodegradable tubular support member is designed in an original manner in order to have:

a biodegradable porous outer surface, enabling cell proliferation and cell vascularization;

a biodegradable non-porous inner surface, in contact with the alimentary bolus in the case where it is an esophageal prosthesis or, more generally, with a bodily fluid circulating in a hollow organ;

a diameter and proportions which are the same as or equivalent to those of the organ to be reconstituted; and

sufficient mechanical properties.

This prosthesis has the advantage of being able to be produced easily from a biocompatible and biodegradable biopolymer and of respecting the anatomical properties of the various organs. Surprisingly, this prosthesis enables an excellent targeted reconstruction of the hollow organ, or portion of hollow organs, to be reconstructed or replaced. The expression “targeted reconstruction” is understood to mean a reconstruction of the organ or of a portion of the organ by cell proliferation within the prosthesis. It is especially surprising that the cells, implanted via the biological material added, are capable of proliferating and are functional in order to allow the reconstruction of the extracellular matrix, in spite of being in the presence of the biodegradable tubular support member, and thus of reconstructuring the replaced portion of the hollow organ. Moreover, the desired mechanical and physiological properties are obtained.

The tubular support member is preferably used in combination with a differentiated or not very differentiated or undifferentiated living material. According to one variant, use is made of a fetal material specific to the organ to be repaired, thus enabling the regeneration of the organ while respecting the structure, the morphology and the function of the organ in question. According to another variant, use is made of living material constituted by at least one portion of the tissue cells of the organ to be reconstructed. These cells are generally the cells that have a good proliferation ability within the porous layer of the invention.

The present invention therefore relates to a complex bioprosthesis composed a) of a biocompatible and biodegradable porous tubular support member, and b) of a not very differentiated or undifferentiated living material and, preferably, a specific fetal material that enables the formation of an ectopic matrix of fetal material after implantation.

One of the objectives of the present invention is to produce a complex bioprosthesis for the regeneration of hollow organs in order to overcome the limitations of the prior art linked to the use of autologous cells or adult tissues. This bioprosthesis enables the regeneration of the entire organ without risks of viral transmissions or graft rejections. (In case of using allogeneic or xenogeneic (donor of strains or species different from those of the receiver) fetal material, the usual measures of immunosuppression or of creating tolerance should be envisaged).

The present invention relates, according to a first aspect, to a prosthesis for promoting the in vivo reconstruction of a hollow organ or of a portion of a hollow organ, characterized in that it comprises:

a biodegradable hollow tubular support member comprising at least one biocompatible and biodegradable polymer material, said support member being constituted of a porous outer layer and an essentially non-porous inner layer; and

a material of living biological origin at the outer surface, and/or within at least one portion of the porous layer of said support member, and/or over the surface of the essentially non-porous layer facing the porous layer, said material of biological origin being chosen in order to allow the in vivo reconstruction of said organ or of said organ portion.

The invention covers variants in which the essentially non-porous layer and the porous layer are constituted of different materials, but also the variants in which these layers are constituted of materials that are identical although their porosity is different.

According to a second aspect, the invention also relates to a process for manufacturing such a prosthesis. This process comprises the preparation of a porous tubular support member comprising an essentially non-porous layer on its inner face and incorporation of a material of biological origin at the outer surface of said tubular support member and/or within it. The process comprises, in particular, the preparation of a biodegradable tubular support member comprising a porous outer layer that enables cell proliferation and an essentially non-porous inner layer (that permits substantially no cell proliferation), and the incorporation of a biological material intended to form a prosthesis at the outer surface, and/or within at least one portion of the porous layer of said support member, and/or on the surface of the essentially non-porous layer facing the porous layer.

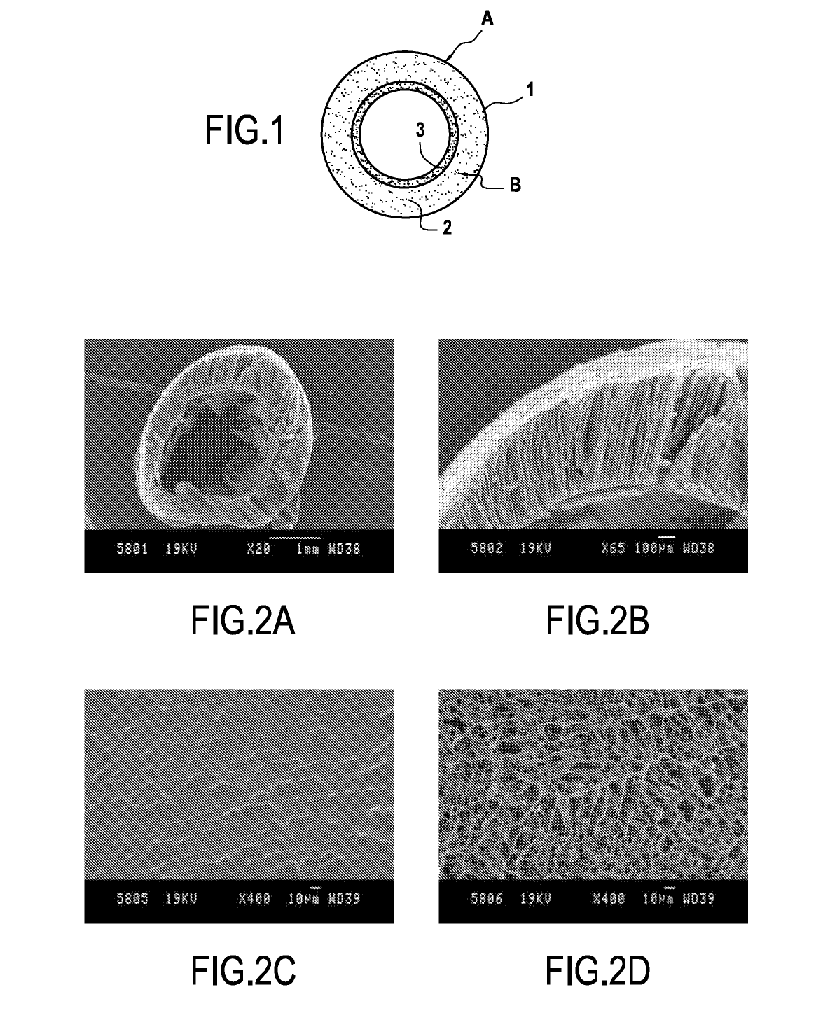

Other features and advantages of the present invention emerge from the detailed description and examples that follow, illustrated by FIGS. 1 to 8.

FIG. 1 is a diagram of a tube of the invention seen in cross section. In FIG. 1, reference 1 denotes the layer on the outside of the tube comprising the biodegradable polymer, reference 2 denotes the space for the development of the cells and of the living organ, and reference 3 denotes the essentially non-porous inner layer. Reference A locates the living material in a first embodiment in which the living material is placed on or fastened to the outer surface of the tube. Reference B locates the living material in a second embodiment in which the living material is placed between the porous outer layer and the non-porous layer.

FIG. 2 composed of FIGS. 2A, 2B, 2C and 2D, presents photographs obtained by scanning electron microscopy that correspond to the porous tube obtained according to example 1.

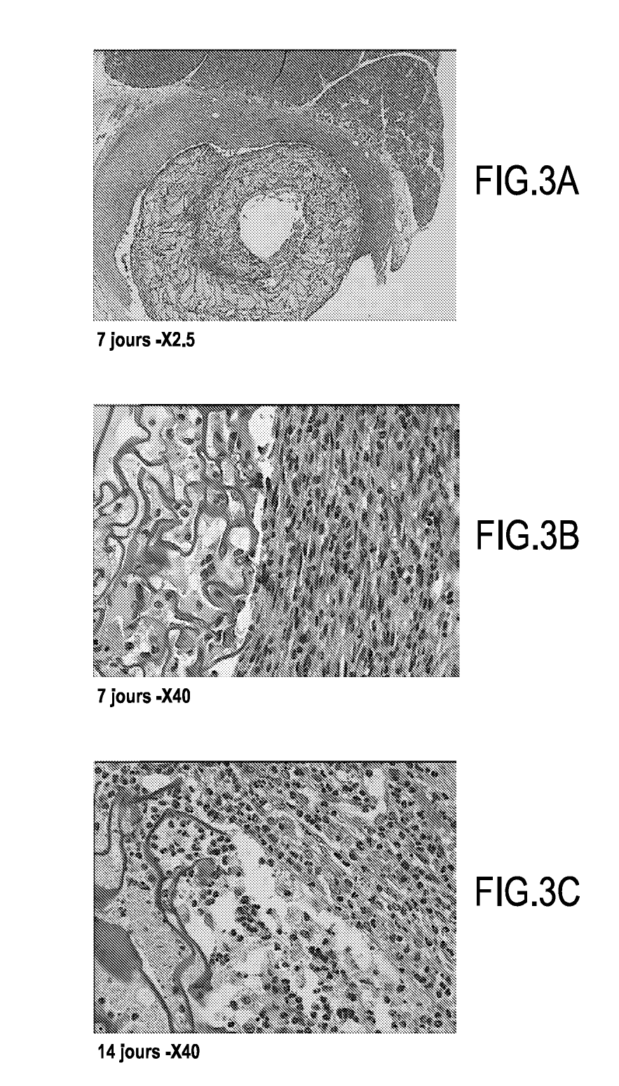

FIGS. 3A, 3B and 3C relate to the steps of integration, then of resorption, of the chitosan material into the body at 7(a,b) and 14(c) days after its implantation.

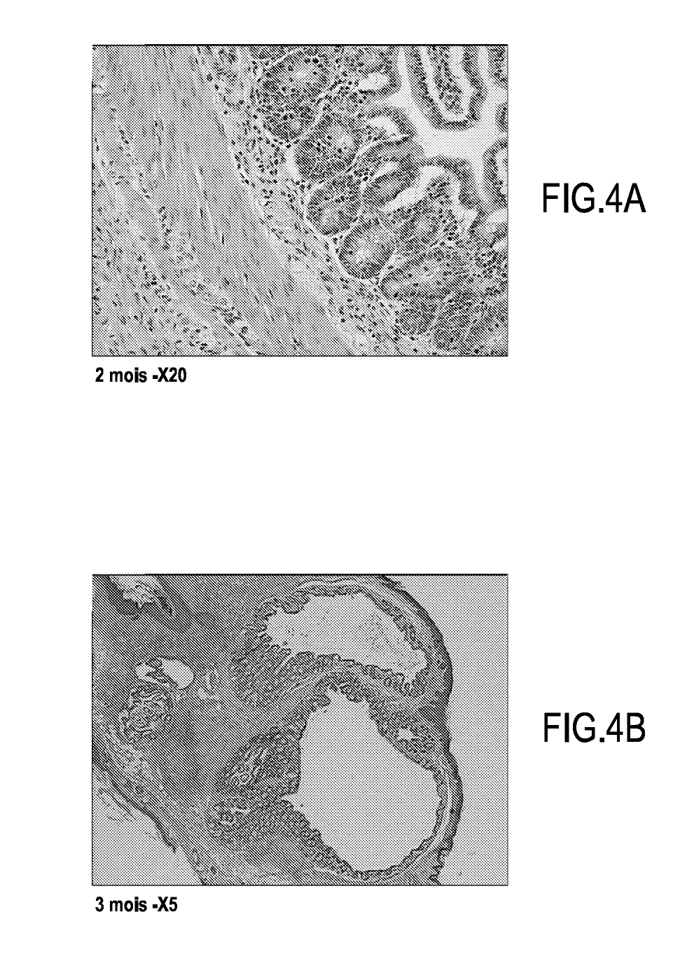

FIGS. 4A and 4B show the ectopic development of a fetal intestine at 2 and 3 months in the presence of a chitosan tube and the disappearance (resorption) thereof.

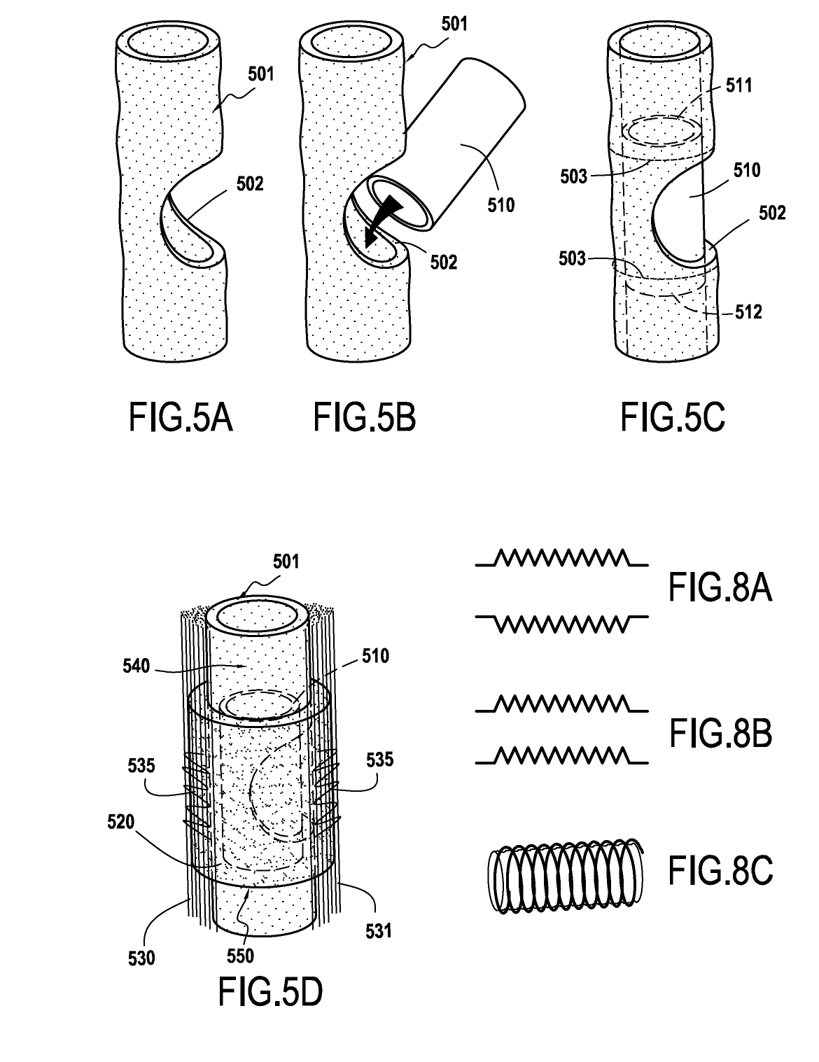

FIGS. 5A, 5B, 5C and 5D schematically represent a method for inserting a variant of the tubular support member of the invention, where the non-porous inner layer and the porous outer layer are physically independent.

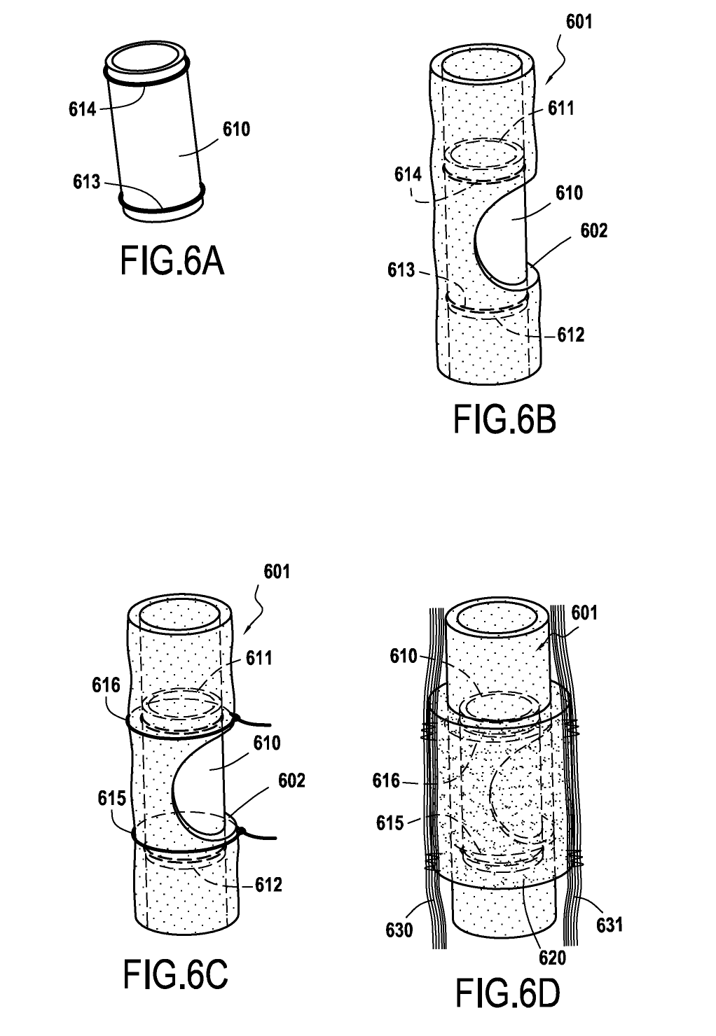

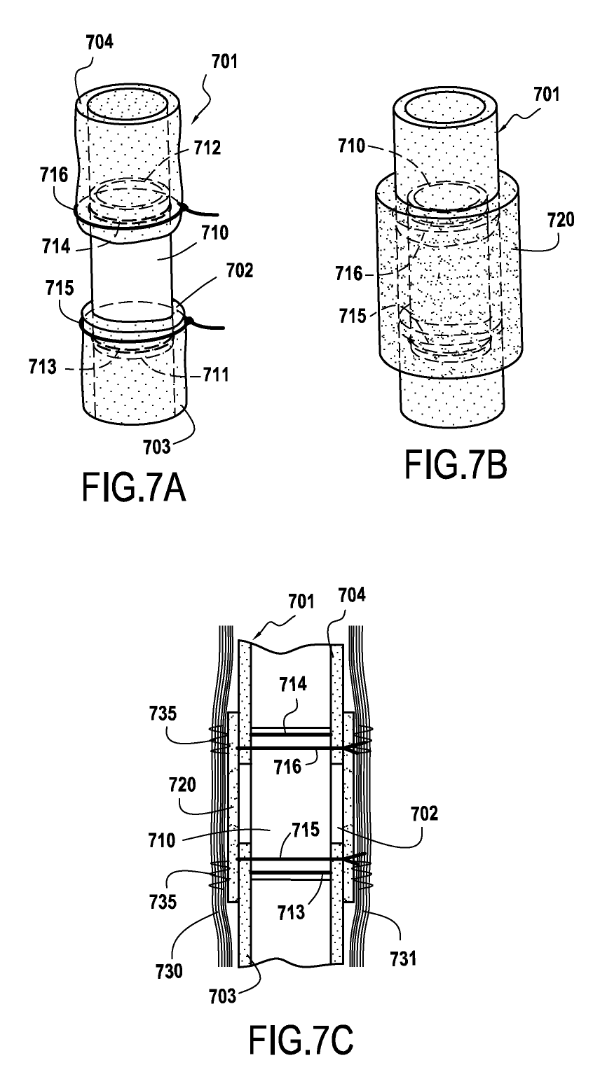

FIGS. 6A, 6B, 6C, 7A, 7B and 7C schematically represent a method for inserting a variant of the tubular support member of the invention, where the non-porous inner layer and the porous outer layer are physically independent.

FIGS. 8A, 8B and 8C schematically represent variants of the invention comprising a means favoring the flexibility of the tubular support member.

The invention relates to a combined bioprosthesis for the regeneration of hollow organs and more particularly for the regeneration of portions of esophagus having a pathology. Other organs may be repaired, replaced or regenerated using the present invention, such as the intestine, the common bile duct, the stomach, the pancreatic duct, the urinary ducts (urethra and ureter), the bladder, the blood vessels, the Fallopian tubes and the uterus. The pathology may be, for example, a cancer. Thus, the invention covers the use of the prosthesis according to the present invention, including all its variants, for replacing a hollow organ, at least one portion of which is affected by a cancer, and in particular when the hollow organ is the esophagus. The invention therefore also covers the methods for the surgical treatment of a cancer of at least one portion of a hollow organ, especially in the case where the cutting out or ablation of an entire section of the hollow organ is necessary. Other pathologies will also be able to benefit from this treatment such as, for example, burns with grave stenosis. Thus, the invention relates to a method for the surgical treatment of a pathology requiring the cutting out or ablation of at least one portion of a section of a hollow tubular organ, characterized in that it comprises the cutting out or ablation of a complete or partial section of a hollow tubular organ, and the positioning, in the vicinity of the area that has been cut out or ablated, of a prosthesis, of a tubular support member, or of a polymer material defined in the invention, including all the variants, for reconstructuring in vivo the cut out or ablated portion.

According to the present invention, the tubular support member is advantageously constructed of a biocompatible and biodegradable polymer. The tube is porous, but combined with an essentially non-porous inert surface at its inner wall. It is advantageous for the porous and essentially non-porous layers to be constituted of the same biodegradable polymer.

According to the present invention, the tissue support member displays sufficient mechanical properties, compatible with the mechanical conditions encountered in vivo.

According to one variant, the tubular support member of the invention comprises a means favoring the flexibility of the support member, especially for improving the resistance to the movement of the reconstructed organ. These means are, for example, an accordion or spiral structure, without being limited thereto.

According to the present invention, the tubular support member is compatible with the biological material, and preferably fetal material.

According to the present invention, the tubular support member is biodegradable in vivo and has a controlled degradation over time giving a temporary support that enables the growth and the proliferation of neo-tissues.

According to the present invention, the tubular support member has specific dimensions and structure in accordance with the anatomy and the function of the organs to be reconstituted. The essentially non-porous layer must ensure the leaktightness of the porous layer and/or of the living material with respect to the biological medium which may be contained in or passed through the organ to be reconstructed (biological liquid, alimentary bolus, etc). The term “leaktightness” is understood to mean the absence of the passage of substances which may deteriorate the functioning of the host, or even an inflammation that cannot naturally be resorbed over time. The essentially non-porous layer may have a thickness between 60 μm and 3 mm, or at most 2.5 or 2 mm. It may also be at least 100 μm thick. The essentially non-porous layer also serves as a support member either for the living material according to one variant of the invention, or for the porous layer optionally containing the living material according to another variant. The non-porous layer may also serve as a guide for the reconstruction of the hollow tubular organ, such as the esophagus.

According to the present invention, the tubular support member may be prepared by methods such as lyophilization, molding, extrusion, solvent evaporation, extraction of pore-forming agents; immersion-precipitation or a combination of these methods.

According to the present invention, the compound bioprosthesis may be used for the repair, the replacement or the regeneration of human hollow organs including the gastrointestinal tract, the digestive, biliary, pancreatic, urinary and genital ducts, and also the blood vessels and nervous tissues.

The complex bioprosthesis of the present invention is constituted of a support member having optimal characteristics for an ectopic growth of biological material and preferably of fetal material. The support member is a biodegradable tubular structure enabling the regeneration of hollow organs such as the digestive, biliary, pancreatic, urinary and genital ducts (esophagus, intestine, stomach, common bile duct, urethra, ureter, bladder, Fallopian tubes and uterus). The concept is not to permanently replace a defective portion with the biodegradable tubular support member but to promote/stimulate the tissue regeneration via the use of a biodegradable tubular support member combined with a transplant of living biological material that is preferably not very differentiated or that is undifferentiated and that is preferably of fetal origin, or combined with at least one portion of the tissue cells of the organ to be reconstructed.

The support member is designed as a tube with a porous outer layer which can stimulate the cell/tissue migration, the vascularization and the regeneration of hollow organs. The inner lumen of the tube is not permeable and may be in contact with the alimentary bolus or any other fluid circulating in the hollow portion of the organ. It also has dimensions and a size in accordance with the organ to be reconstituted.

As explained previously, the tubular support member which serves as a support for the living biological material may be constituted of various polymers, provided that they will be able to be used to obtain a tube having dimensions similar to those of the organ or of the organ portion to be reconstituted and sufficient mechanical properties (elasticity, strength and flexibility without reduction of the shape and of the lumen) and also a porosity suitable for ensuring a good adhesion of the biological material, and preferably of the fetal material, during its growth in vivo and ensuring a normal circulation of the fluid normally circulating in the hollow organ.

More specifically, the porosity must be of sufficient size to allow cell infiltration and colonization by the blood vessels and also the growth of the biological material, and preferably of the fetal material.

The pores are preferably interconnected in order to allow cellular interactions, the diffusion of oxygen and of metabolites.

The porosity is preferably continuous throughout the thickness of the tube, up to its inner surface.

The inner diameter must be adapted to the size of the duct to be reconstructed. The choice of the outer diameter is less important. However, the flexibility of the tube, which must be maintained, must be taken into account.

The inner layer or surface of the tube must be impermeable and non-porous, so as to enable the leaktightness of this tube to chyme in the case of digestive ducts (for example the esophagus and the stomach), to gases in the case of a respiratory duct (for example, the trachea) or to any other fluid in the case of other organs, so as to prevent the passage of bacteria and viruses.

Moreover, this inner surface is constituted of an essentially non-porous layer that substantially prevents cell proliferation in order to avoid a non-targeted cell proliferation which will eventually seal the lumen of the tubular support member.

Generally, the mechanical strength of the tube is, preferably, sufficient to prevent the crushing of the tube and to maintain the lumen (internal diameter) ensuring the passage of air or of the alimentary bolus or of any other fluid depending on the organ to be reconstructed.

The polymer material preferably has a degradation, over time, necessary for the regeneration of the organ. It must also be biocompatible so as not to induce cell toxicity, an inflammatory reaction or a rejection reaction and it should also be compatible with the biological material, and preferably fetal material.

Furthermore, the polymer material must be able to be easily sterilized.

As explained previously, the support member is advantageously constituted of chitosan, which is a readily available material and which may result, via a simple process, in all the advantages explained above. However, a large number of other polymers known for their biodegradability and biocompatibility properties could be chosen.

More specifically, the polymer material is chosen from the group constituted of chitosan, chitin, a chitin-glucan copolymer and from derivatives or copolymers thereof, these polymers being optionally combined with at least one other biocompatible and biodegradable polymer.

Various other biocompatible and biodegradable polymers could be used in combination with chitosan, chitin or the derivatives or copolymers thereof defined above, especially in order to vary one or more of their properties, such as their cell proliferation ability, their mechanical strength, their degree of swelling on contact with the biological medium of the host bordering the prosthesis, their deformability, their degradation rate, their compressibility, elasticity, suppleness, flexibility, etc.

Use could in particular be made of biopolymers, in particular biopolymers chosen from the group constituted of glycosaminoglycans (GAGs), in particular hyaluronan, chondroitin sulfate or heparin, collagens, alginates, dextrans and mixtures thereof.

It is also possible to choose biodegradable and biocompatible synthetic polymers, in particular chosen from the group constituted of synthetic biodegradable polyesters such as homopolymers and copolymers based on lactic acid, glycolic acid, epsilon-caprolactone and p-dioxanone or else any other natural polyester such as those from the poly-hydroxyalkanoate family such as homopolymers and copolymers based on hydroxybutyrate, hydroxyvalerate, polyorthoesters and polyurethanes.

Use will preferably be made of chitosan or a polymer material containing it.

Chitosan is manufactured by deacetylation of chitin, the various possible sources of which are well known. These are the shell of crustations (crabs, prawns and lobsters mainly), cephalopods endoskeletons, arthropods cuticles diatomaceous earths and cell walls of fungi. Preferably polymers of fungal origin will be chosen, due to the hypoallergenic nature thereof, the constant and easily traceable quality thereof and the almost unlimited and completely renewable source thereof, moreover allowing a reuse of by-products of the agro-food and biotechnology industry. Chitosan may advantageously be produced from the process described in patent application WO 03068824 to Kitozyme.

Chitosan preferably has a degree of deacetylation and a molecular weight that are chosen so as to ensure an optimal degradation rate. It has, for example, been shown that the degradation rate of chitosan depends strongly on its molecular weight and on its degree of deacetylation, in the sense that the lower the molecular weight and the degree of deacetylation, the faster the degradation. Consequently, the control of the porosity is important, support members with larger pore sizes and higher porosities degrade more rapidly.

Chitosan, when it is chosen for preparing tubes that are used as a support member, may be combined with other biodegradable polymers, for example another glycopolymer such as chitin or chitin-glucan. Methods for preparing these polymers or copolymers are described in patent applications by Kitozyme (WO 03068824, FR 05 07066 and FR 06 51415).

As explained previously, the porosity of the tubular support member is essential for allowing the attachment and growth of the biological material, and preferably the fetal material, after incorporation of the prosthesis in vivo.

This porosity must be sufficient to allow blood cells at least, and optionally some graft cells, to pass through. The diameter of the pores of the porous portion is therefore greater than 10 μm and preferably between 10 and 200 μm.

The inner diameter and the thickness of the tube constituting the support member are adapted to those of the hollow organ that it is desired to reconstruct.

The dimensions, in particular the thickness, of the polymer depends on the targeted physical properties, these properties having to guarantee an elasticity and a strength in connection with the nature of the organ to be reconstructed. This thickness also depends on the diameter of the tube and on the nature of the organ to be reconstructed. It is understood that, in any case, the internal diameter of the tube is given by the diameter of the organ to be reconstructed.

The living material may be placed at the surface of, or in, the outer and porous layer of the tube, and optionally held in place by a woven fabric wound around the latter. Another possibility is to place the living material between the impermeable inner surface of the tube (essentially non-porous layer) and its porous surface. In this case, the porous layer and the essentially non-porous layer may not be firmly attached and are designed independently. They may therefore be physically independent. The non-porous layer may be a film or a second non-porous tube. The expression “essentially non-porous” is understood to mean the fact that the cells or the biological material associated with the biodegradable polymer do not colonize, completely or a little, and preferably do not colonize, the non-porous layer.

The addition of biological material to or into the biodegradable tubular support member is preferably carried out in vivo or just before the resection.

According to one embodiment, the biodegradable hollow tubular support member is implanted in order to replace at least one portion of a hollow organ, then the material of living biological origin is introduced at the surface of, or in, the porous layer, or at the surface of the essentially non-porous layer facing the porous layer. The proliferation of the material of living biological origin therefore takes place in vivo. This allows a very advantageous reconstruction of the hollow organ or portion of hollow organs to be reconstructed or replaced.

According to a second embodiment, the biological material is added to the support member just before resection to avoid a step of culturing of the biological material.

According to a third embodiment, the biodegradable tubular support member is implanted without biological material. The support member is then colonized by the host cells.

According to a fourth embodiment, the tubular support member is produced as two physically independent and separate (that is to say independently manipulable) parts, a first part comprising the porous layer, and the second part comprising the essentially non-porous layer. Within this context, the non-porous layer is placed on the inside of the hollow tubular organ to be reconstructed, then the porous layer is placed on the outside of this organ.

These embodiments make it possible, in particular, to avoid in vitro seeding and cell culturing conditions, but also enables a saving in time and in production costs. On the other hand, it is not necessary to constitute a cell bank. In these advantageous embodiments, the prosthesis is intended for an in viva incorporation of the material of living biological origin. The cell colonization carried out in vivo is very good and the reconstruction of the portion or all of the organ replaced is permitted.

The biological material of human origin may be of cell origin (excluding embryonic stem cells) and preferably germinal stem cells, including the cells taken from a fetus of more than 8 weeks, in particular from a fetus between 8-10 weeks, or from the umbilical cord after birth. Preferably, the living material used is not very differentiated or is undifferentiated and, preferably, of fetal origin. It may also be constituted by the proliferative cells of the tissue to be reconstructed.

Fetal stem cells (taken from a fetus of 8-10 weeks) will preferably be used compared to adult stem cells, as they are more abundant. The adult stem cells will preferably be taken from the organ to be reconstructed (stomach, intestine, uterus, bladder, blood vessels).

The cells may be cells from at least one animal, especially from a mammal, or from at least one human being.

The fetal material may be either an organ, or an organ segment, or an emulsion of cells. This fetal material is advantageously in a wet and viscous form, so as to be able to be spread over a surface, that of the tube to which it will have to adhere or to which it will have to be attached, forming a sort of network dressing. Another alternative consists of the use of stem cells, the differentiation of which could be controllable.

The thickness of the layer deposited will advantageously be from 0.1 to 1 mm, but could also be greater. A person skilled in the art understands that the thickness of this layer depends mainly on the nature of the organ and on the nature of its receiver (human or animal).

The proportions of polymer and of biological material, and preferably of fetal material, may also vary in large proportions as a function of the nature of the organ to be reconstructed.

The advantages of the use of fetal materials are:
a high degree of survival of the transplant even in the absence of vascularization (owing to the diffusion of nutrients while waiting for colonization by the vessels of the host).
The fetal organs are sufficiently undifferentiated to allow a high capacity for growth and for regeneration of material organs while being sufficiently differentiated to avoid any error in their development and their growth (no deviant development observed for a material that is fetal in origin). The differentiation of fetal materials is easier and enables, for example, a better control of the differentiation than during the use of stem cells.
The fetal material does not contain infectious agents and therefore reduces the risks of viral transmissions.

As described previously, the invention also comprises the process for preparing the prosthesis of the invention.

This process comprises the preparation of a porous tubular support member comprising an essentially non-porous layer on its inner face and the incorporation at the surface of this tubular support member and/or within it, of a material of fetal origin.

As explained previously, porous tubular support members, and especially chitosan-based support members are already known. These support members may be used for the preparation of the prostheses of the invention.

Generally, the technique for producing polymer-based tubes having a porous structure and a non-porous (impermeable) inner layer is well known.

Lyophilization is one method which is well known for the preparation of porous materials. Its principle is based on freezing a solution in order to induce crystallization of the solvent.

The solvent is then removed by vacuum sublimation in order to create pores in place of the solvent crystals. This technique combines the following advantages:
simplicity of use;
possibility of controlling the porosity and the diameter of the pores by playing with the processing parameters and formulation parameters (cooling rate, concentration of the polymer solution, etc.);
various types of geometries are attainable: porous membranes, 3D support members, beads or tubes; and
industrial extrapolation can be easily envisaged.

As described in the publication: “Porous chitosan scaffolds for tissue engineering” (S. V. Madihally, H. W. T. Matthew, Biomaterials 20 (1999), 1133-1142), porous chitosan tubes are prepared by lyophilization, by freezing a solution of chitosan in the annular space between two concentric tubes (made of silicon or polytetrafluoroethylene), the chitosan solution is injected into this space and the whole assembly is frozen by direct contact, namely with dry ice at −78° C. (as described in the article). The outer tube is then removed and the assembly is lyophilized. Carried out according to this method, the tube is completely porous throughout its thickness, including the outer surface and the inner surface (or luminal surface).

In order to obtain tubes characterized by a non-porous luminal wall, various solutions could be used, the same authors describe a method based on the prior covering of the inner silicon tube with a film of chitosan. This chitosan film may be obtained by dipping the tube into a solution of chitosan and by gelling it by rapid immersion in a 30% aqueous ammonia solution and by then leaving it to dry. The film could also be prepared directly by simple evaporation of the solvent, as is carried out in example 1, which is more advantageous.

Once rehydrated in an aqueous medium, the support members described above will rapidly swell and end up by dissolving again, due to the presence of soluble chitosan acetate within the lyophilized structure. Furthermore, the dissolution of the support members may be avoided by neutralizing the samples via immersion either in a solution of NaOH, or in a series of alcohols of decreasing concentration (S. V. Madihally, H. W. T. Matthew, Biomaterials 20 (1999), 1133-1142).

Use will advantageously be made of chitosan concentrations between 1 and 10% in acetic acid for the preparation of porous tubes by lyophilization.

Besides the thermally induced phase separation or lyophilization technique, other techniques intended to form pores are well known for the preparation of porous supports.

Mention will be made of: the extraction of pore-forming salts, a supercritical fluid (supercritical CO₂) foaming, and also more recent methods such as the technique known by the expression “solid free-forming” which consists in constructing contours of three-dimensional objects, but most of these methods do not allow a good control of the porosity and generate weakly connected porous structures.

To make the lumen of the tube non-porous, a non-porous tube may be inserted inside the porous tube or else, the hollow non-porous tube may be surrounded by a porous membrane constituting the outer porous part of the tubular support member. In these cases, the porous and non-porous layers may then be physically independent. In this variant, a ridge located at each end of the non-porous tube may be provided so as to improve the sealing of the non-porous tube/esophagus join. This ridge may be produced by means of yarns previously placed on the non-porous tube, by an overthickness of the material of the tube or of a material different from the non-porous tube. This ridge may also facilitate the attachment of the tube to the esophagus.

A porous chitosan tube having sufficient mechanical strength is obtained by lyophilization of chitosan solutions. The solvents used for dissolving the chitosan are organic and inorganic acids such as formic acid, lactic acid, succinic acid, hydrochloric acid, gluconic acid and preferably acetic acid. They may be used for making the chitosan tubes.

Ideally, the chitosan solutions are prepared by dissolving chitosan at concentrations of 1-10% in an aqueous solution of acetic acid.

Ideally, the chitosan used as a starting material for the design of bioprostheses is of fungal nature and is obtained by deacetylation of chitin extracted from fungi, for example according to the processes described in the patent applications by Kitozyme indicated above.

Chitosan advantageously has a degree of acetylation and a molecular mass chosen so as to obtain an optimal degradation rate that is in keeping with the regeneration rate of the organ to be regenerated.

The neutralization of the chitosan support member is advantageously attained by a sodium hydroxide treatment in order to obtain a support member that is compatible with physiological conditions. It is preferable to treat with a 1% NaOH solution.

The chitosan support member may be sterilized by γ-irradiation or ethylene oxide methods, or by autoclaving.

The present invention covers a biodegradable tubular support member, as defined previously, intended for the reconstruction of at least one portion of a hollow organ.

The present invention also covers a porous biocompatible and biodegradable polymer material for the surgery for repairing a hollow organ of tubular shape, said polymer material being intended to form the porous layer of a biodegradable hollow tubular support member comprising or constituted of a porous outer layer and an essentially non-porous inner layer.

Advantageously, the biodegradable hollow tubular support member is positioned in fine so that the porous outer layer is positioned on the outer surface of the hollow organ, and the essentially non-porous inner layer is positioned on the inner surface of the hollow organ.

According to one embodiment, the polymer material of tubular shape comprises a distal end and a proximal end, said proximal end being intended to be positioned at one end of a completely or partially severed hollow organ, and said distal end being intended to be positioned at another end of the completely or partially severed hollow organ.

This arrangement makes it possible to replace or reconstruct a complete or partial section of the hollow organ.

It is easily understood that the expression “end of the hollow organ” is understood in the broad sense and relates to the case of a partial section of a hollow organ, a portion of the tissue of the organ facing another portion of the tissue possibly being linked geometrically by a straight line that passes through the severed portion of the tissue, said straight line not passing through the lumen of the hollow organ.

The present invention covers a method of cell proliferation, especially for reconstructing at least one portion of a hollow organ, the steps comprising the production of a biodegradable tubular support member comprising a porous outer layer and an essentially non-porous inner layer, and the seeding of cells or of the tissue implant at the outer surface, and/or within at least one portion of the porous layer of said support, and/or on the surface of the essentially non-porous layer facing the porous layer, under conditions that allow their proliferation within the porous layer.

Other objectives, features and advantages of the invention will appear clearly to a person skilled in the art after reading the explanatory description which refers to examples which are given solely by way of illustration and which should not in any way limit the scope of the invention.

The examples are an integral part of the present invention and any feature that appears novel relative to any prior art based on the description taken in its entirety, including the examples, is an integral part of the invention functionally and generally.

Thus, each example has a general scope.

### EXAMPLES

### Example 1

### Manufacture of Chitosan Porous Tubes

A chitosan of plant origin produced by KitoZyme, characterized by its viscosity-average molecular weight of 42 K and a degree of acetylation of 11%, is put into a solution in acetic acid (1%) in an amount of 5% (weight/volume).

The porous support member of tubular shape is manufactured by lyophilization of the chitosan solution, previously injected using a syringe into the annular space formed by two concentric tubes of different diameters. The assembly is frozen by direct contact in liquid nitrogen for 15 minutes. The outer tube is then removed and the lyophilization of the assembly is continued for 24 h. After drying, the inner tube is in turn removed and the tube obtained is analyzed by scanning electron microscopy.

FIGS. 2A, 2B, 2C and 2D represent the photographs obtained and illustrate the structure of the tube. FIG. 2A is a transverse cross section which clearly shows that the porosity is obtained over the whole of the thickness of the tube. FIG. 2B shows this porous structure particularly well. The inner surface of the tube is illustrated by FIG. 2C which shows that the pores do not open into the lumen of the tube. FIG. 2D, which gives the appearance of the outer layer of the tube, shows that the pores, on the contrary, open very clearly onto the outside of the tube.

### Example 2

### Histology After Subcutaneous Implantation of Porous Tubes and Membranes in Rats and Mice

Chitosan porous support members in the form of tubes, prepared by lyophilization of chitosan solutions in acetic acid were first neutralized by treatment with a solution of NaOH (to eliminate the acid residues), then sterilized either by exposure for 20 min in 96° alcohol followed by washing in a saline buffer for 5 min, or by autoclaving. 10 BALBc mice and 5 Fisher rats received the chitosan implants (membranes or tubes) subcutaneously, one in each ear.

Tubes made of paraffin and polyethylene were used as controls.

At various time intervals (7, 14 and 62 days), external biometric analyses and histological analyses were carried out.

The results shows that in all the animals, the chitosan implants are well tolerated (FIG. 3A) and infiltrated by the surrounding cells and tissues already after 7 days (FIG. 3B). A moderate inflammatory reaction is observed and no implant has induced a rejection reaction. The chitosan implants begin to degrade between weeks 1 to 4 (FIG. 3C) and are completely resorbed after 62 days.

In conclusion from this example, the chitosan prostheses are biocompatibie, allow the infiltration of neighboring cells and tissues and only lead to a very limited inflammatory reaction during their degradation.

### Example 3

### Subcutaneous Implantation of Porous Tubes and Membranes, Associated with Fetal Material, in Mice

Fetal intestine was taken from mice fetuses after 15 to 20 days of intrauterine development and implanted into subcutaneous pouches made in the ocular pavilion of host mice (10 mice); this fetal material being combined with a chitosan implant in the form of tubes just before implantation by covering the outer surface of the chitosan tubular support member with the fetal material. The strain of the donor and of the receiver is identical (syngeneic transplantation) in order to avoid immunobiological rejection reactions.

In this example, the chitosan tubes were sterilized by treatment with alcohol for 30 to 40 minutes before being washed with a sterile saline solution for 5 minutes then 25 minutes in order to remove any residue of alcohol.

After 2 months (FIG. 4A) and 3 months (FIG. 4B), the intestine implants show an excellent development, the chitosan support member being completely degraded at the end of this period. After 2 months (FIG. 4A) a cross section through the fetal implant shows the development of a normal intestine similar to adult intestine and exhibiting all its features (villi structure) in the presence of the chitosan tube which, itself, is completely resorbed. This experiment therefore shows that the chitosan implants are compatible with the development of syngeneic fetal transplants of digestive organs, in this case the intestine. The intestinal lumen is visible in FIG. 4B.

Histological sections taken in the lungs, liver and kidneys in the host at periods of 3 months demonstrate the absence of an inflammatory reaction and of a harmful effect with respect to these organs.

### Example 4

### Simulation of an Esophageal Bioprosthesis

Combined prostheses composed of a chitosan porous tube and fetal intestinal material (the fetal material being placed either on the outside of the tube, or between the porous layer and the inner non-porous surface of the tube) were implanted longitudinally between the neck muscles in rats without disturbing the esophagus. This experiment shows the ability of the chitosan tube to be colonized by the fetal intestinal material and to withstand the movements of the neck.

### Example 5

### Replacement of an Esophageal Segment by the Use of a Chitosan Porous Tube Covered by Syngeneic Fetal Esophagus or Intestine

Segments of fetal intestine were collected between 14 and 18 days of intrauterine development in rats and positioned around chitosan porous tubes.

After resection of an esophageal segment of 0.5 to 1 cm in length in the neck of the rat, the chitosan tube with the fetal material is fastened to the two severed ends of the esophagus of the rat in such a way that the joins between the prosthesis and the organ are hermetic or leaktight. The same experiment is repeated with fetal esophageal material.

### Example 6

### Tolerance After Implantation in the Neck of the Rat of a Chitosan Hollow Tubular Support Member

The hollow tubular support member from example 6 is composed of a first non-porous tube prepared according to the process described in WO 2007042281 from a chitosan sample from example 1, and characterized by an internal diameter of 1.5 mm and an external diameter of 2.5 mm. The non-porous tube is surrounded by a porous membrane prepared according to a conventional lyophilization process from chitosan from example 1, thus constituting the outer porous layer of the tubular support member. The non-porous tube and the membrane are both sterilized (autoclaving or immersion in a disinfecting alcohol-containing solution for 15 to 20 minutes) then rinsed in a physiological solution (0.9% NaCl) for at least 20 minutes.

The anaesthetized rat is placed on a suitable support on its back, stretched out so as to present the anterior face of the neck. A median section of the skin from the level of the thyroid cartilage to that of the suprasternal notch is made, the subcutaneous muscles are incised, the pretracheal muscles slit longitudinally and in the interstices the chitosan tube surrounded by the chitosan porous membrane is placed longitudinally. The muscular and subcutaneous planes are closed up by suturing.

After sacrificing on day 90 (3 months), the animal did not exhibit any macroscopic impairment of the external appearance of the internal organs. The anatomopathological study showed an almost complete disappearance of the membrane, conservation of the tube, well surrounded by fibrous tissues, no macroscopic nor microscopic impairment of the internal organs and of the tissues surrounding the tube and the membrane.

### Example 7

### Implantation of a Hollow Tubular Support Member in a Partially Severed Esophagus

FIGS. 5 and 6 serve to schematically support this example. They in no way constitute a representation of the actual detail and proportions, which are not respected.

An anaesthetized rat was subjected to a longitudinal section of the skin of the neck followed by prominently displaying the tracheal and the esophagus (501), a portion of which (502) (around ⅔ of the circumference) was severed (FIG. 5A). The non-porous tube (510) from example 6 was then introduced inside the esophagus (501) via the portion of the severed organ (502) (FIG. 5B), then attached using threads (503) previously placed around the esophagus (501) and tightening the tube+esophagus assembly at the ends of the tube (511, 512) (FIG. 5C). The porous membrane (520) from example 6 is then wound around the esophagus+tube, then attached to the adjacent muscle tissues (530, 531) by means of a suture stitch (535) (FIG. 5D). The living material (540) is therefore placed in situ between the porous outer face (520) and the non-porous inner face (510) of the tubular support (550).

The postoperative decline is without local complication (dehiscence of the sutures, abscess, superficial infection). The animal experiences a few difficulties in drinking and feeding for 10 days and loses weight, then the situation improves rapidly. The rat is sacrificed after 35 days. The anatomopathological observation reveals that after 35 days the rat has regained its initial weight. The internal organs have a normal appearance.

The esophagus is Ieaktight, non-stenotic and reconstituted. No local abscess or leakage (of the alimentary bolus, bodily fluid, etc.) was observed. The prosthesis therefore made it possible to reconstitute a leaktight connection with the esophagus.

The analysis of the histological sections shows that the prosthesis has definitely disappeared from the site (it has not been found in any part of the digestive tube, therefore has been resorbed or digested), that the esophagus and the neighboring tissues have a normal appearance. Some residues of membrane in an area slightly infiltrated by the living material were found in the vicinity of the cervical esophagus.

Example of one possible procedure for implantation of the materials:
1. preparation of the non-porous chitosan tube (610) and of the porous membrane (620) after sterilization thereof either using an autoclave or by immersion in a disinfecting alcohol-containing solution for 15 to 20 min:
rinsing the tube (610) using a sterile physiological solution for 20 min at least;
rinsing the porous membrane (620) with the same physiological solution (0.9% NaCl) for the same period of time;
placing 2 marker and connection suture threads (613, 614) that form a sort of ridge around the two ends (611, 612) of the tube (610) (FIG. 6A). This ridge forms an overthickness at the ends of the tube (610) and may also facilitate the attachment of the tube to the esophagus.
2. prominently displaying the tracheal and the esophagus (601), dissection of the esophagus,
3. partial section of the esophagus (601) (section over around ⅔ of the circumference) with or without ablation revealing a cavity (602);
4. insertion of one end of the tube (610), and fastening in the esophagus (601) by means of the threads (615, 616) surrounding each free edge of the esophagus (601) severed so that the ridge formed by the thread (613, 614) is on the inside of the organ, insertion and fastening of the other end in a similar manner (FIGS. 6B and 6C);
5. winding of the porous membrane (620) (sterilized and rinsed in the same way as the tube) on the outside of the esophagus (601) and of the tube (610) (at their interface). A suture stitch is placed between the ends of the membrane (620) and the adjacent tissues (630, 631) in order to fasten the membrane (620) (FIG. 6D). The threads 615 and 616 may also be used to fasten the porous membrane (620);
6. closing the operation wound by planes.

### Example 8

### Implantation of a Hollow Tubular Support Member in a Completely Severed Esophagus

A longitudinal section of the skin of the neck followed by prominently displaying the tracheal and the esophagus of an anesthetized rat was carried out. The cervical esophagus is completely severed at mid-height. The non-porous tube from example 6 is then introduced inside the esophagus then fastened using threads previously positioned around each free edge of the severed esophagus in order to clamp the tube+esophagus assembly at the ends of the tube and position the tube so as to reconstruct the tube formed by the esophagus. The porous membrane from example 6 is then wound around the esophagus and the tube then fastened to the adjacent muscle tissues using a suture stitch. The living material is then placed between the porous outer face and the non-porous inner face of the tubular support member.

The postoperative decline is without local complication (dehiscence of the sutures, abscess, superficial infection). The animal experiences a few difficulties in drinking and feeding.

The observations were carried out 1, 3 and 6 days after the operation. The tube-esophagus join was leaktight. No local infection or abscess were observed. The histological sections show a small local inflammatory reaction and confirm the presence of the tube and of fragments of membrane.

Example of one possible procedure for implantation of the materials:

The first operating steps carried out for example 8 are identical to steps 1 and 2 from example 7. Steps 3 and 4 differ by the fact that the esophagus undergoes a no longer partial but indeed total section. They are described as follows:
3. complete section of the cervical esophagus (701), with or without ablation, at mid-height, thus creating a total section (702);
4. insertion of one end (711) of the non-porous tube (710), and fastening in the distal part (703) of the esophagus (701) using threads (715, 716) surrounding each free edge (703, 704) of the severed esophagus (701), so that the ridge formed by the thread (713, 714) on the tube is inside the organ (701), insertion and fastening of the other end (712) in the proximal segment (704) in a similar manner (FIG. 7A);
5. winding of the porous membrane (720) on the outside of the esophagus (701) and of the tube (710) (FIG. 7B);
6. one or more suture stitches or dots of adhesive (735) are placed between the ends of the membrane and the adjacent tissues (730) in order to fasten the membrane (720) (FIG. 7C); and
7. suturing of the wound in two planes—muscular plane and cutaneous plane.

### Example 9

### Embodiment Variant of the Hollow Tubular Support Member of the Invention

A tubular support member according to the present invention produced in accordance with the preceding examples may comprise a portion having sequential variations in cross section so as to form an accordion-type structure that makes it possible to improve the flexibility of the tube, and therefore the resistance to the movement of the host, and swallowing too. It is possible, for example, to implement the protocol from example 1 by using two concentric annular spaces each having a portion comprising sequential variations of cross section instead of the annular space formed by two concentric tubes of different diameters. It is also possible to implement the protocol for preparing a non-porous tube from example 6 in order to prepare a non-porous tube having a structure, in particular, of accordion type that makes it possible to improve the flexibility of the tube.

FIGS. 8A and 8B schematically represent two variants of this portion of tube.

Likewise, the concentric annular spaces may have various shapes, such as for example in order to have variations in thickness in the form of a spiral. FIG. 8C schematically represents a variant of a spiral.

### Example 10

### Graft of Fetal Intestine or of Esophagus Alone

Step 1: (4 rats) preparation of the animals as in example 6, but the tube is replaced by a segment of intestine taken from rat fetuses of the same strain aged 17 days.

Step 2 (1-2 months later): the neck is reopened, the cyst formed by the growing fetal esophagus or intestine is opened and rinsed, shaped so as to form a longitudinal tube of dimensions equal to those of the esophagus without damaging its vascular connections with the body, the esophagus of the receiver is prominently displayed, a segment is resected after positioning marker threads on the edges of the section of each side, then the “fetal” tube is sutured to each end of the esophagus via a continuous suture 6.0. If possible, biological adhesive is placed on the sutures in order to reinforce the sealing thereof. Closing of the operating wound in two planes.

This example shows the feasibility of a graft of living material of fetal origin for the reconstruction of a tubular organ.

### Example 11

### Plastic Surgery of Esophageal Segments with Tube Graft Combination

Method: example 10 is reproduced, but a non-porous tube made of chitosan (prepared according to WO 2007/042281) is fastened to the inside of the “fetal” tube in order to give it greater rigidity and strength, for the time needed for the sutures to be strengthened and for the body to “repair” the esophageal circular defect. The tube can then be removed.

1-27. (canceled)

28. A prosthesis for promoting the in vivo reconstruction of a hollow organ or of a portion of a hollow organ, wherein said prosthesis comprises:
a biodegradable hollow tubular support member comprising at least one biocompatible and biodegradable polymer material, said support member being constituted of a porous outer layer and an essentially non-porous inner layer; and
a material of living biological origin at the outer surface, or within at least one portion of the porous layer of said support member, or over the surface of the essentially non-porous layer facing the porous layer, said material of biological origin allowing the in vivo reconstruction of said organ or of said organ portion, said material of biological origin.

29. The prosthesis as claimed in claim 28, wherein said polymer material is selected from the group consisting of chitosan, chitin, and from derivatives or copolymers thereof optionally combined with at least one other biocompatible and biodegradable polymer.

30. The prosthesis as claimed in claim 29, wherein said at least one other biocompatible and biodegradable polymer is a biopolymer selected from the group consisting of glycosaminoglycans (GAGs), hyaluronan, chondroitin sulphate, heparin, collagens, alginates, dextrans and mixtures thereof.

31. The prosthesis as claimed in claim 29, wherein said at least one other biocompatible and biodegradable polymer is a biocompatible and biodegradable synthetic polymer selected from the group consisting of synthetic biodegradable polyesters, homopolymers based on lactic acid, copolymers, glycolic acid, epsilon-caprolactone, p-dioxanone, a natural polyester, a poly-hydroxyalkanoate family, hydroxybutyrate-based homopolymers, hyd roxyvalerate-based homopolymers, polyorthoester-based homopolymers, polyurethane-based homopolymers, hydroxybutyrate-based copolymers, hydroxyvalerate-based copolymers, polyorthoester-based copolymers, polyurethane-based copolymers, and any mixture thereof.

32. The prosthesis as claimed in claim 28, wherein said polymer comprises or is constituted of chitosan.

33. The prosthesis as claimed in claim 32, wherein said chitosan is obtained by deacetylation of chitin.

34. The prosthesis as claimed in claim 28, wherein the diameter of the pores of the porous portion is greater than 10 μm.

35. The prosthesis as claimed in claim 28, wherein the internal diameter and the thickness of the tubular support member are adapted to those of said hollow organ.

36. The prosthesis as claimed in claim 28, wherein said hollow organ is an organ selected from the group consisting of a digestive duct, biliary duct, urinary duct, genital duct, blood duct, oesophagus, intestine, stomach, common bile duct, pancreatic duct, urethra, ureter, bladder, Fallopian tubes, uterus and blood vessels.

37. The prosthesis as claimed in claim 36, wherein said hollow organ is the oesophagus.

38. The prosthesis as claimed in claim 28, wherein said material of biological origin comprises tissue cells of the organ to be reconstructed or cells that are not very differentiated or that are undifferentiated.

39. The prosthesis as claimed in claim 28, wherein said material of biological origin is a material of fetal origin.

40. The prosthesis as claimed in claim 39, wherein said material of fetal origin is an organ, an organ segment, or an emulsion of cells of fetal origin.

41. The prosthesis as claimed in claim 40, wherein said material of fetal origin is in a wet and viscous form, so as to improve its adhesion to the surface of or within said support.

42. A process for manufacturing a prosthesis as claimed in claim 28, wherein said process comprises the preparation of a biodegradable tubular support member comprising a porous outer layer that enables cell proliferation and an essentially non-porous inner layer, and the incorporation of a biological material intended to form a prosthesis at the outer surface, or within at least one portion of the porous layer of said support member, or on the surface of the essentially non-porous layer facing the porous layer, said material of biological origin.

43. The process as claimed in claim 42, wherein the preparation of the outer porous layer is carried out by lyophilization.

44. A biodegradable tubular support member as defined in claim 28, for the reconstruction of at least one portion of a hollow organ.

45. A porous biocompatible and biodegradable polymer material for the surgery of a hollow organ of tubular shape, said polymer material being intended to form the porous layer of a biodegradable hollow tubular support member comprising or constituted of a porous outer layer and an essentially non-porous inner layer.

46. The polymer material as claimed in claim 45, wherein the biodegradable hollow tubular support member is positioned in fine so that the porous outer layer is positioned on the outer surface of the hollow organ, and the essentially non-porous inner layer is positioned on the inner surface of the hollow organ.

47. The polymer material as claimed in claim 45, wherein the polymer material is positioned in tubular form and comprises a distal end and a proximal end, said proximal end being intended to be positioned at one end of a completely or partially severed hollow organ, and said distal end being intended to be positioned at another end of the completely or partially severed hollow organ.

48. A non-porous biocompatible and biodegradable polymer material for the surgery of a hollow organ of tubular shape, said polymer material being intended to form the non-porous layer of a biodegradable hollow tubular support member comprising or constituted of a porous outer layer and an essentially non-porous inner layer.

49. A method for the regeneration of a hollow organ, said method comprising placing a tubular support member as claimed in claim 44, or a biodegradable hollow tubular support member comprising or constituted of a porous outer layer and an essentially non-porous inner layer, wherein a porous biocompatible and biodegradable polymer material forms the porous layer of the biodegradable hollow tubular support member.

50. The method as claimed in claim 49, wherein the organ is at least one portion of the esophagus that exhibits a pathology.

51. The method as claimed in claim 49, wherein said organ is selected from the group consisting of intestine, common bile duct, stomach, pancreatic duct, urinary ducts, urethra, ureter, bladder, blood vessels, Fallopian tubes, and uterus.

52. The prosthesis as claimed in claim 49, wherein said hollow organ comprises a portion affected by any pathology or by a cancer.

53. A method for the surgical treatment of a pathology requiring the cutting out or ablation of at least one portion of a section of a hollow tubular organ, wherein said method comprises cutting out or ablation of a complete or partial section of a hollow tubular organ, and positioning, in the vicinity of the area that has been cut out or ablated, a prosthesis as claimed in claim 28, a biodegradable hollow tubular support member comprising at least one biocompatible and biodegradable polymer material, said support member being constituted of a porous outer layer and an essentially non-porous inner layer; or a biodegradable hollow tubular support member comprising or constituted of a porous outer layer and an essentially non-porous inner layer, wherein a porous biocompatible and biodegradable polymer material forms the porous layer of the biodegradable hollow tubular support member, for reconstructing in viva the ablated portion.

54. The method as claimed in claim 53, for a surgical treatment of a cancer or for a burn with grave stenosis.
